(19) United States
(12) Patent Application Publication
(10) Pub. No.: US 2025/0040894 A1
(43) Pub. Date: Feb. 6, 2025

(54) PERSONALIZED CHEST ACCELERATION DERIVED PREDICTION OF CARDIOVASCULAR ABNORMALITIES USING DEEP LEARNING

(71) Applicant: Northwestern University, Evanston, IL (US)

(72) Inventors: Ethan Johnson, Evanston, IL (US); Mahmoud Ebrahimkhani, Evanston, IL (US); Michael Markl, Evanston, IL (US)

(21) Appl. No.: 18/792,492

(22) Filed: Aug. 1, 2024

Related U.S. Application Data
(60) Provisional application No. 63/517,817, filed on Aug. 4, 2023.

(51) Int. Cl. A61B 5/00; A61B 5/0205; A61B 5/11; A61B 5/352; G16H 10/60; G16H 50/20

(52) U.S. Cl. CPC A61B 5/7267; A61B 5/0205; A61B 5/1103; A61B 5/726; A61B 5/7282; G16H 10/60; G16H 50/20; A61B 5/352

(57) ABSTRACT

A system and method for predicting cardiovascular function is presented. The method includes accessing physiological measurement data and patient data from a patient. The method also includes accessing a neural network trained to predict cardiovascular function data using the physiological measurement data and patient data.

### CROSS-REFERENCE TO RELATED APPLICATIONS

This application claims the benefit of U.S. Provisional Patent Application Ser. No. 63/517,817, filed on Aug. 4, 2023, and entitled “PERSONALIZED CHEST ACCELERATION DERIVED PREDICTION OF CARDIOVASCULAR ABNORMALITIES USING DEEP LEARNING,” which is herein incorporated by reference in its entirety.

### BACKGROUND

Medical imaging techniques such as echocardiography, computed tomography (CT), or magnetic resonance imaging (MRI) play an integral role in the clinical assessment of cardiovascular diseases. These tools facilitate the evaluation of both functional and morphological abnormalities in the cardiovascular system.

Echocardiography, phase contrast MRI (PC-MRI), four-dimensional (4D) flow MRI, and cardiac gated CT imaging are routinely utilized to identify and monitor functional changes, which may be prevalent in patients suffering from heart valve, aortic, or pulmonary diseases. These techniques enable time-resolved measurements of blood velocity, offering a comprehensive insight into the temporal and spatial evolution of cardiovascular hemodynamics. Consequently, crucial cardiovascular flow metrics, like peak systolic velocity, regurgitation fraction, and wall shear stress can be retrospectively derived from these measurements. Furthermore, the precise morphological characterizations of the cardiovascular system can be acquired using techniques such as CT. This allows for the assessment of significant parameters such as aortic dimensions and the identification of venous occlusions.

However, despite the advancements and efficiencies in the development of these imaging protocols, their application in a clinical setting presents several challenges. The sophisticated technology required, the need for skilled operators for image acquisition, and the computational demands for advanced hemodynamic analysis make these techniques both costly and time-consuming. Therefore, there is a pressing need for a method that is cost-effective, efficient, and non-invasive. This method should be capable of providing an accurate evaluation of cardiovascular function metrics, akin to those obtained through clinical imaging. Moreover, it should facilitate the diagnosis of cardiovascular abnormalities, such as valve dysfunction and aortic dilatation.

### SUMMARY OF THE DISCLOSURE

The present disclosure addresses the aforementioned drawbacks by providing a system and method for predicting cardiovascular function. In some configurations, a method for predicting cardiovascular function from physiological measurement data using machine learning is provided. The method includes using a computer system to access physiological measurement data acquired from a subject and patient data corresponding to the subject. The method further includes accessing a machine learning model that was trained on training data to quantify cardiovascular function based on physiological measurement data and patient data. Using the computer system, the physiological measurement data and patient data can be input into the machine learning model in order to generate cardiovascular function data as an output, which can be presented to a user. The cardiovascular function data may include a classification prediction or a prediction of the peak systolic velocity.

In another aspect, the present disclosure provides a method for training a machine learning model for evaluating cardiovascular function. The machine learning model may use ground truth data and physiological measurement data. The method includes accessing ground truth data using a computer system. The ground truth data may indicate a measure of cardiovascular function of each patient within a group of patients. The method may further include accessing physiological measurement data and patient data from the group of patients using the computer system. The patient data may indicate at least one of demographic or clinical data from each patient within the group of patients. The method further includes training a machine learning model using the ground truth data, the physiological measurement data, and the patient data. The machine learning model may include a multilayer perceptron and a convolutional neural network to predict a cardiovascular function of a patient that is based on physiological measurement data and patient data acquired from the patient.

### BRIEF DESCRIPTION OF THE DRAWINGS

FIG. 1 provides a flow chart of an example process in accordance with some aspects of the present disclosure.

FIG. 2(a) shows an example of cardiac magnetic resonance imaging data in accordance with some aspects of the present disclosure.

FIG. 2(b) shows an example of a wearable seismocardiography (SCG) device in accordance with some aspects of the present disclosure.

FIG. 2(c) shows an example ECG waveform in accordance with some aspects of the present disclosure.

FIG. 2(d) shows an example of acceleration data in the x-direction measured by the wearable device of FIG. 2(b) in accordance with some aspects of the present disclosure.

FIG. 2(e) shows an example of acceleration data in the y-direction measured by the wearable device of FIG. 2(b) in accordance with some aspects of the present disclosure.

FIG. 2(f) shows an example of acceleration data in the z-direction measured by the wearable device of FIG. 2(b) in accordance with some aspects of the present disclosure.

FIG. 2(g) shows an example of electrocardiogram (ECG) data used to segment SCG data in accordance with some aspects of the present disclosure.

FIG. 2(h) shows an example of one segment of SCG data in accordance with some aspects of the present disclosure.

FIG. 3(a) shows an example of raw and denoised SCG data in accordance with some aspects of the present disclosure.

FIG. 3(b) shows an example of SCG data after removing outliers in accordance with some aspects of the present disclosure.

FIG. 3(c) shows an example of outlier SCG data in accordance with some aspects of the present disclosure.

FIG. 3(d) shows an example of denoised SCG data in accordance with some aspects of the present disclosure.

FIG. 3(e) shows an example of a scalogram corresponding to the SCG data of FIG. 3(d) in accordance with some aspects of the present disclosure.

FIG. 4(a) shows an example of a machine learning architecture used to predict a measure of cardiovascular function in accordance with some aspects of the present disclosure.

FIG. 4(b) shows an example of a machine learning architecture used to classify cardiovascular function in accordance with some aspects of the present disclosure.

FIG. 5 is a block diagram of an example magnetic resonance imaging (“MRI”) system that can implement the methods described in the present disclosure.

FIG. 6 is a block diagram of an example system that can implement the methods of the present disclosure.

FIG. 7 is a block diagram of example components that can implement the system of FIG. 6.

FIG. 8(a) shows an example of predicted peak systolic velocity (V max) compared to ground truth Vₘₐₓ in accordance with some aspects of the present disclosure.

FIG. 8(b) shows a Bland-Altman plot for the Vₘₐₓ data plotted in FIG. 8(a).

FIG. 8(c) shows example results of a classification model predicting cardiovascular function in accordance with some aspects of the present disclosure.

FIG. 8(d) shows an example confusion matrix for model classification of cardiovascular function in accordance with some aspects of the present disclosure.

FIG. 9(a) shows a second example of predicted Vₘₐₓ compared to ground truth Vₘₐₓ in accordance with some aspects of the present disclosure.

FIG. 9(b) shows a Bland-Altman plot for the Vₘₐₓ data plotted in FIG. 9(a).

### DETAILED DESCRIPTION

Before any aspects of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The present disclosure provides a comprehensive, broad-based system and method for evaluating cardiovascular function or status, including cardiovascular function, valvular dysfunctions, and abnormal cardiovascular blood flow dynamics in the heart and surrounding vessels, typically found in patients with various health conditions. This proposed method harnesses the potential of a collection of non-invasively integrated sensors capable of acquiring multimodal physiological signals. The sensors could include, but are not limited to, microelectronic systems proficient in recording seismocardiography (SCG), electrocardiography (ECG), photoplethysmography (PPG), pulse transit time (PTT), or blood pressure (BP) data.

An aspect of this disclosure is the incorporation of machine learning techniques to predict cardiovascular functions using data from physiological measurement data, such as SCG and ECG, alongside patient demographic data. In some embodiments, the method includes accessing a computer system to process patient-specific multimodal physiological data, such as SCG and ECG, as well as corresponding demographic data, including but not limited to height, weight, sex, and age and clinical factors (e.g., hypertension, history of heart disease, diabetes, or history of other cardiovascular conditions).

In other embodiments, the method can be extended to integrate additional data sources, such as continuous or cuff blood pressure, blood-oxygen saturation, and more. A neural network can be used that has been rigorously trained on extensive ground-truth data to accurately quantify cardiovascular function based on physiological measurements, such as SCG and patient data. With the assistance of the computer system, both sensing data (SCG, ECG, etc.) and patient demographic data can be fed into the neural network. The output of this network can be a detailed assessment of the cardiovascular function. This could range from data representing specific classifications, such as valve insufficiency, to continuously-valued quantities like stroke volume, vessel size, and vessel stiffness, or blood flow velocities.

By providing an output that could be effectively interpreted by the user, the proposed method forms an invaluable tool for predicting various aspects of cardiovascular function, enabling enhanced preventative, diagnostic, and therapeutic strategies.

The present disclosure further introduces a comprehensive method for training a machine learning model geared towards assessing cardiovascular function. This model employs a set of data including, but not limited to, ground truth data and physiological measurement data, such as SCG data.

The method initiates by accessing the ground truth data via a sophisticated computer system. This ground truth data serves as an accurate measure of cardiovascular function for each individual in a designated patient group. The scope of this method is not restricted to using ground truth data alone; it extends to accessing multimodal physiological data acquired from an array of integrated sensors as well as patient-specific data from the same group of patients, via the computer system. The patient data may encompass demographic or clinical data from each patient within the group but is not limited to these parameters.

In some aspects, the machine learning model can incorporate various machine learning architectures, such as a multilayer perceptron and a convolutional neural network. The trained model has the capability to predict a patient's cardiovascular function based on physiological data (e.g., SCG data) and patient data. However, the machine learning model is not limited to these data sources alone. This emphasizes the versatility and comprehensive nature of the proposed method in evaluating cardiovascular function, paving the way for personalized and accurate cardiovascular assessments.

The present disclosure offers advanced systems and methods for the non-invasive assessment of cardiovascular flow dynamics. This is achieved by integrating multiple data sources, such as physiological measurement data and patient data. Physiological measurement data may include but is not limited to seismocardiography (SCG), electrocardiography photoplethysmography (PPG), pulse transit time (PTT), blood pressure (BP), or a combination thereof. The patient data may encompass demographic information, patient medical history, medication use, and other clinical factors. Compared to conventional clinical imaging techniques such as MRI, CT, and echocardiography, this integration of multiple sources proves to be a more cost-effective and efficient approach.

The disclosed system and method include the application of deep learning to train models based on multimodal physiologic sensor measurements. Here, clinical imaging data serves as the ground truth. The model, which may include various architectures such as convolutional neural networks (CNN), is capable of estimating several measures of cardiovascular function. These may include peak systolic velocity (Vₘₐₓ), ejection fraction (EF), stroke volume (SV), aortic dimensions, aortic wall stiffness, classification of valve conditions, and others.

The model further incorporates demographic attributes like body mass index, gender, age, etc., by utilizing such attributes as inputs for an additional network, such as a multi-layer perceptron. This network can be complemented with data about clinical factors and history, including prior imaging results, genetic testing information, smoking history, medication usage, and more.

By predicting cardiovascular dynamics through a combination of physiologic measurements, such as those obtained from a cost-effective wearable SCG/ECG device, and from easily accessible patient data, it is possible to diagnose abnormalities before prescribing a comprehensive and costly imaging test such as cardiac MRI. This approach aids in reducing the burden on the healthcare system and potentially guides future cardiac MRI exams, thereby minimizing unnecessary recurrent testing.

Physiological measurement data may include SCG data. SCG signals are non-invasive measures of cardiac vibrations obtained at the chest surface, which may be referred to as precordial vibrations. These vibrations are associated with heart mechanical activities such as cardiac contractions, valve closures, and changes in blood flow velocities and momentum. For example, the atrial systole has been shown to result in a low-frequency SCG wave component, while the ventricular systole produces a high-amplitude wave, and the first and second heart sounds generate two high-frequency vibrations. Additionally, simultaneous SCG and electrocardiogram (ECG) acquisition has revealed that the timing of various physiological events, such as the opening and closure of the mitral and aortic valves and their rapid filling or ejection, correspond to various features in the SCG waveforms. However, SCG pulses are highly susceptible to inter-subject variations due to differences in factors such as body mass index, gender, age, and other demographic and health attributes, making it difficult to derive consistent clinical inferences. Additionally, the location of the SCG device on the chest can impact the shape of the recorded waveforms. Lastly, SCG vibrations have relatively low amplitudes and are easily contaminated by subject motion artifacts or respiratory movements, which can lead to misinterpretation of diagnostic features.

In some implementations, the disclosed systems and methods can include mitigating these potential confounds by processing the physiological measurement data. For instance, the physiological measurement data can be pre-conditioned to remove or otherwise reduce interference and/or spurious data points from the physiological measurement data.

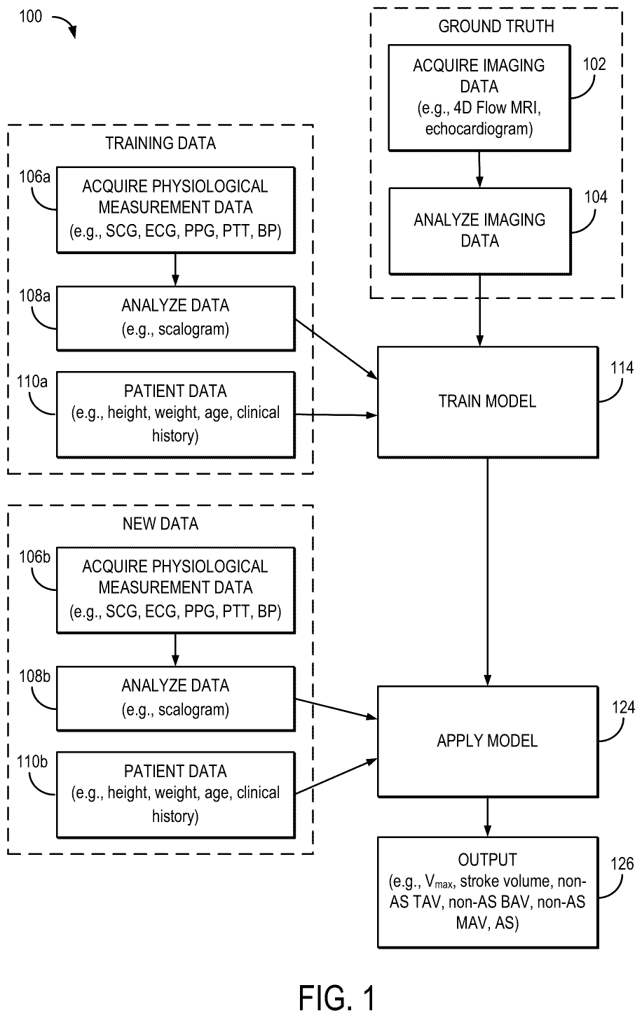

Referring to FIG. 1, a flowchart demonstrates the general steps of a process 100. The process 100 occurs in two main phases: training a model using ground truth data and training data, and applying the model in inference using new data. Training and applying the model may be performed on two groups of subjects or patients, which may or may not include some of the same subjects within each group. For example, during a validation phase, training and applying the model may be repeated several times after adjusting the subjects in each group. In general, after model training and validation are complete, the model can be applied for new subjects without the need for ground truth (e.g., 4D flow MRI) data. Advantageously, the model may be re-trained in order to predict new parameters of interest based on the desired clinical application, eliminating the need to redesign the underlying networks and enhancing its utility in the face of evolving medical scenarios. For example, the model may be trained to predict ejection fraction, stroke volume, aortic valve regurgitation fraction, wall shear stress, heart malformations, mitral valve dysfunction, etc.

In the first phase, ground truth data is accessed or generated. The ground truth data can be accessed from memory by a computer system. The ground truth data may also be generated by analyzing imaging data. For example, imaging data may be acquired in block 102. Such imaging data may include MRI data, such as 4D flow data acquired using phase-contrast. The imaging data may also include echocardiographic imaging, including Doppler flow imaging, CT imaging data, or data acquired with other imaging techniques. Block 102 may include acquiring imaging data using an imaging system, such as an MRI or ultrasound system, or may include accessing stored imaging data. Acquiring data in block 102 may also include image processing or reconstruction. For example, block 102 may include reconstruction of raw k-space data and optional pre-processing steps, such as eddy current correction, velocity anti-aliasing, parallel reconstruction, motion correction, or other data correction or quality control.

As a non-limiting example, the MRI data may include 4D flow data with a spatial resolution between 1-3 mm³, time resolution of 30-40 ms, and velocity encoding (venc) of 150-375 cm/s. The data may be acquired on a 1.5 T, 3 T, 7 T, or other field strength MRI system. The data may be acquired with free breathing and navigator respiration control or with repeated breath holds. Prospective or retrospective cardiac gating may be used to acquire the full desired volume of the heart, such as the thoracic aorta.

The imaging data may be analyzed in block 104 to provide a ground truth for model training. Block 104 may include known methods for analyzing cardiac MRI data to generate the desired metrics or classification of the cardiovascular function. For example, block 104 may generate flow metrics, such as peak systolic velocity (Vₘₐₓ), ejection fraction (EF), stroke volume (SV), aortic dimensions, or aortic wall stiffness. Block 104 may also classify the cardiovascular function, such as the presence or severity of aortic valve disease. Metrics can also be generated for other valves and vessels, such as the mitral valve, pulmonary valve, or pulmonary veins. Such measurements can characterize valve status or blood flow (e.g., velocity, volume, etc.).

As a non-limiting example, analysis in block 104 may include segmentation of the aorta. For example, a deep learning tool may be applied to automatically derive a 3D segmentation of the thoracic aorta. The segmentation may be used to mask the 4D flow velocity data. Analysis may also include delineating the ascending aorta (AAo) using known techniques, for example by manually delineating from its root to the branching vessels by placing a plane proximate to the branching vessels and perpendicular to the centerline. Hemodynamic parameters such as peak systolic velocity (Vₘₐₓ) may be acquired using volumetric analysis of all voxels in the AAo or other vessel of interest. Outlier rejection may optionally be applied to acquire more accurate results.

Block 104 may also include classification of heart conditions, such as aortic stenosis (AS), aortic valve condition (e.g., normal tricuspid aortic valves (TAV), bicuspid aortic valves (BAV), and mechanical aortic valves (MAV)), aortic valve regurgitation, heart malformations, mitral valve insufficiency, cardiac output insufficiency (i.e., healthy or unhealthy ejection fraction or stroke volume) etc. For example, classification may be achieved by a radiologist or other expert, or by other known methods using 4D flow data. The classification may also include a measure of the severity of the heart condition, such as mild, moderate, or severe.

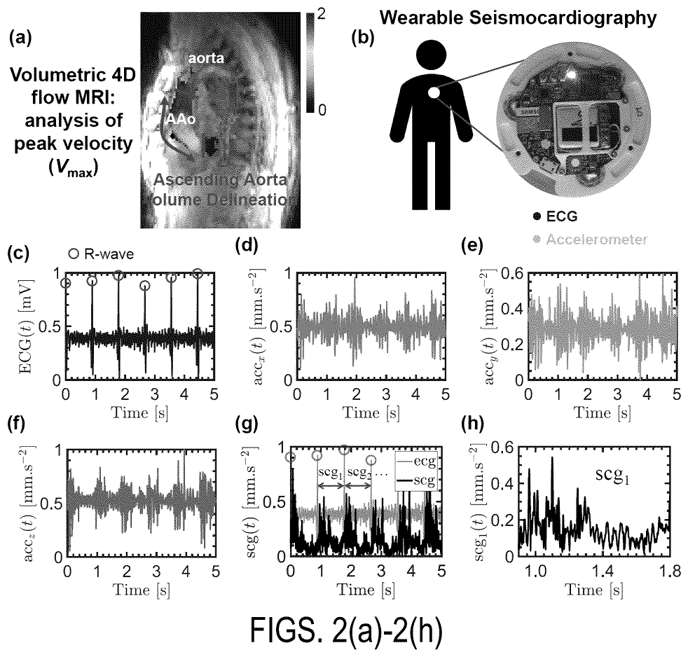

FIG. 2(a) shows an example of 4D flow data, masked to obtain voxels within the aorta and delineated to obtain voxels within the AAo. The color bar indicates the velocity of blood flow. For example, peak systolic velocity Vₘₐₓ may be calculated using the velocity measurements within the whole AAo with outlier rejection.

Referring again to FIG. 1, non-invasive cardiovascular data may be acquired for each subject in block 106a. Acquiring non-invasive cardiovascular data may include accessing measurement devices, such as a wearable SCG device and ECG electrodes or accessing stored data using a computer system. For example, the non-invasive cardiovascular data may include measurements of SCG, ECG, blood oxygen saturation, blood pressure, etc. Some or all of the physiological measurement data may be acquired using an integrated multimodality wearable measurement device. Such a device may integrate several sensors to simultaneously measure SCG data, ECG data, PPG data, PTT data, BP data, or a combination thereof. Such data can be measurements resolved over the entire cardiac cycle or scalar parameters that summarize an aspect of cardiovascular function (such as peak systolic/diastolic blood flow velocities, blood pressure, systolic and diastolic venular volumes, ejection fraction, stoke volume, etc.). The training data may include patient data in block 110a, which will be discussed further below.

As a non-limiting example, the SCG data may include chest acceleration data acquired by a wearable cardiac sensor. FIG. 2(b) shows an example of a custom SCG sensor, which can be placed on the chest of a subject. For example, the sensor may include a MEMS accelerometer. The accelerometer can measure accelerations in one or more directions. For example, acceleration can be measured in three orthogonal directions (e.g., x, y, and z). As a non-limiting example the sensor may have a sampling rate of 1 kHz with 2 μm/s² sensitivity, for example. The SCG device may be placed on the chest of the subject. For example, it may be placed on the sternum while the subject is in the supine position. SCG data may be recorded for a time period of 1-30 seconds, or longer. For example, the SCG data may be acquired for a 5-second period for each subject.

Referring again to FIG. 1, block 106a may additionally include ECG data, which may be referred to as cardiac or chest voltage signals. Such ECG data measures electrical potential on the chest's surface. The ECG data may be acquired by ECG sensors that may be integrated with the SCG sensor or may be placed separately on the chest. The ECG data may be simultaneously acquired with the SCG data such that the SCG data can be processed according to the ECG data. Thus, the ECG data and SCG data can be combined to interpret the SCG data in the context of the cardiac cycle. For example, the SCG data may be gated by ECG data.

FIG. 2(c) shows an example of ECG data used for gating. For example, the R-waves may be determined using the ECG data so that the SCG acceleration recordings may be beat-by-beat time-referenced to the R-waves. In this way, a single SCG pulse may be used from each heartbeat. The SCG data may include acceleration data in several directions. For example, FIGS. 2(d)-2(f) show an example of acceleration data acquired in x, y, and z. As another non-limiting example, the directional data may be combined to produce a single SCG pulse. For example, the x, y, and z components of the acceleration data may be combined as the root sum of squares according to SCGᵢ(t)=\sqrt{acc_{i,x}²+acc_{i,y}²+acc_{i,z}²}, where acc_{i,x} represents the acceleration in x of the i^{th} heartbeat, as shown in FIG. 2(g), for example. Additionally or alternatively, the model may be trained using acceleration measured in a single direction, or independent accelerations measured in several directions (e.g., acc_{i,x}, acc_{i,y}, and acc_{i,z} prior to combining). FIG. 2(g) also demonstrates how the SCG signal was gated by the ECG data in order to separate the SCG signal into individual SCG signals for each R-R period, as shown in FIG. 2(h), for example.

Referring again to FIG. 1, block 106a may also include pre-processing of the training data. For example, the SCG data may be processed to remove noise and outliers. As a non-limiting example, the SCG signal may be processed using a high-pass filter to remove low-frequency artifacts. The filter may be a minimum-order elliptic filter with an infinite impulse response (IIR) function. As a non-limiting example, the filter may have a stop-band attenuation of 60 dB, a stop-band frequency of 8.4 Hz, a pass-band frequency of 10 Hz, and a pass-band ripple of 0.1. The transition may be tapered using a Blackman window, for example.

Pre-processing in block 106a may also include denoising to improve the signal-to-noise ratio. For example, wavelet denoising may be used. As a non-limiting example, denoising may be achieved using the false discovery rate (FDR) with a hard thresholding rule and a symlet wavelet with four vanishing moments (‘sym4’). A level-dependent noise estimate may be used, and the number of levels of decomposition may be calculated as the integer part of log₂(N), where N is the number of signal points.

Outliers can be removed from the training data in block 106a. As a non-limiting example, outliers can be identified using the signal quality index (SQI), which measures the quality of the signals calculated based on the distance of each SCG from a reference signal. Removing outliers can help ensure that the resulting signals are of high quality and accurately represent the subject's physiology. The SQI can be determined for the i^{th} heartbeat as:

$${SQI}_{i} = exp ( - \frac{𝒟 ( {SCG}_{i} ( t ) , S \hat{C} G ( t ) )}{ℒ ( {SCG}_{i} ( t ) , S \hat{C} G ( t ) )} )$$

where SĈG(t) represents the point-wise average of all SCGs for the subject, [symbol] (SCGᵢ(t), SĈG(t)) represents the distance between SCGᵢ(t) and SĈG(t), and [symbol] (SCGᵢ(t), SĈG(t)) represents the length of SCGᵢ(t) and SĈG(t). [symbol] (SCGᵢ(t), SĈG(t)) can be calculated using dynamic time warping (DTW), allowing the calculation of the minimum Euclidean distance between two SCG signals even if they are of different lengths. [symbol] (SCGᵢ(t), SĈG(t)) may be calculated by stretching or compressing the signals to match their lengths. In this way, a small distance [symbol] indicates a high-quality signal, while a large distance indicates a poor-quality signal. Outliers can be removed by setting a threshold of the SQI score. For example, the top 95% of signals may be maintained, while any signals with an SQI below the 5% threshold can be considered outliers and removed.

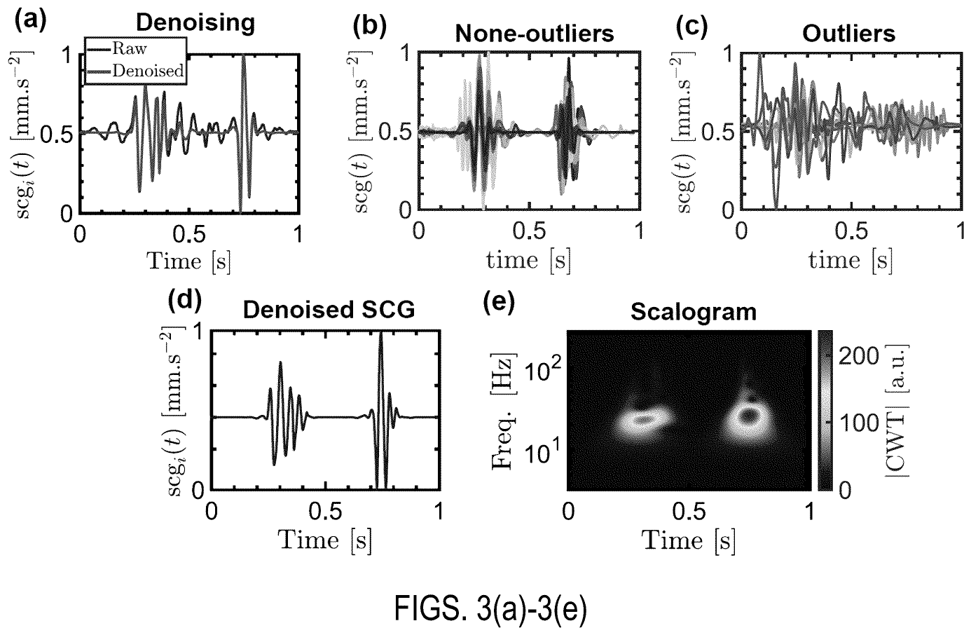

FIGS. 3(a)-3(c) show an example of SCG data pre-processing, which may be completed in block 106a, for example. The pre-processing includes denoising and removing outliers. FIG. 3(a) shows a comparison of raw and denoised SCG signal. FIGS. 3(b)-3(c) show SCG signals for a single subject after separating them into good SCG signals, shown in FIG. 3(b), and outlier signals, shown in FIG. 3(c). The outlier signals are highly fluctuating and do not resemble a typical SCG data.

Referring again to FIG. 1, after pre-processing SCG and ECG data, block 108a may include analyzing the non-invasive cardiovascular data. Analyzing the SCG data can provide a time-frequency representation used for training the model. For example, block 108a may include generating scalograms using a continuous wavelet transform (CWT). Other transforms that include time- and frequency-resolved decomposition may be used as well, such as a short time Fourier transform.

As a non-limiting example, the CWT may be applied by defining a CWT filter bank composed of 48 filters per octave using the analytic Morse wavelet. The filter amplitudes can be normalized so that the peak magnitude of all passbands is equal to a chosen value, such as 2. The highest passband frequency may be designed such that the magnitude falls to half the peak value at the Nyquist frequency.

A scalogram may be produced for each SCG using the absolute value of the CWT coefficients. An example of a scalogram and its corresponding denoised SCG is provided in FIGS. 3(d)-3(e). In this example, two main features in the SCG pulse, which occur between 0 and 0.5 seconds and between 0.5 and 1 second, appear at the same locations in the scalogram. In addition, the frequency components of these features, which lie between 10 and 100 Hz, are shown on the y-axis. These frequency values were directly estimated from their corresponding wavelet scales.

The scalograms can be converted to images, and their size may be reduced. For example, the scalogram images may be interpolated by representing each pixel as the weighted average of its nearest neighbors. As a non-limiting example, the images may be reduced to 256 pixels×256 pixels images using bicubic interpolation using neighborhood size of 4 pixels×4 pixels. This step can allow for easy visualization and analysis of the time-frequency characteristics of the SCG signals.

Referring again to FIG. 1, the scalograms may be used as a part of the training data as an input to block 114. The training data may additionally include patient data acquired in block 110a. Acquiring patient data may include accessing external measurement devices, such as blood pressure cuff, scale, etc., accessing stored patient data by a computer system, or inputting patient data with a computer system or user interface by a user. Patient data may include the patient's demographic data, such as age, height, weight, body mass index, sex, etc. In addition, the patient data may include known patient medical history, such as use of tobacco or other substances, known conditions (e.g., hypertension, diabetes, prior cardiovascular events, arrhythmias, etc.), medication usage (e.g., beta blockers, statins, etc.), genetic information acquired from genetic testing, family history, etc. The patient data may be used as another input for training the model in block 114.

The model details will be provided in further detail below. In general, ground truth data, physiological measurement, and patient data are provided for each subject, during the training phase. The ground truth data may be provided using costly and time-consuming imaging techniques (e.g., MRI) and may include a measure of a cardiovascular metric, such as Vₘₐₓ, SV, EF, etc. or a classification of a cardiovascular condition, such as AS, BAV, MAV, TAV, aortic valve regurgitation, heart malformations, etc.

Once the model is trained in block 114, costly and time-consuming ground truth data is not required to apply the model. Instead, new data can be provided using cost-effective and accessible methods, such as SCG and ECG, in blocks 106b, 108b, and 110b. Blocks 106b, 108b, and 110b follow methods described for 106a, 108a, and 110a, respectively. However, as the new data is acquired for each subject, costly MRI data is not required for those subjects. The trained model can be applied to the new data in block 124 to provide a clinical output in block 126. For example, the output may include a measure of Vₘₐₓ or a classification of aortic valve status (e.g., AS, BAV, MAV, TAV), based on how the model was trained in block 114.

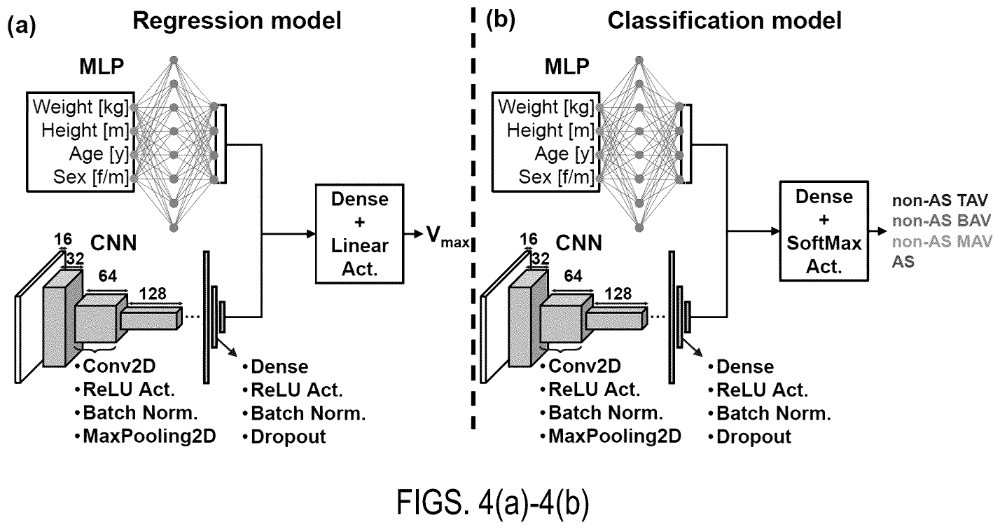

FIGS. 4(a)-4(b) provide example model details for regression and classification deep learning models that may be used to train the network as described above. For example, the regression model may be used to estimate a measure of cardiovascular function, such as peak systolic velocity (Vₘₐₓ) in the AAo, stroke volume, or left ventricular ejection fraction. The classification model may be used to classify cardiovascular conditions, such as aortic valve condition (e.g., non-AS TAV, non-AS BAV, non-AS MAV, AS, aortic valve regurgitation), heart malformations, other heart valve conditions (e.g., mitral valve insufficiency), aortic dissection status, etc. In either case, the SCG scalograms or other physiological measurement data may be used as input data for a convolutional neural network (CNN) model.

The disclosed system provides a tunable or adjustable computational network, commonly known as a neural network, with inputs matched to the preprocessed measured signals. As a non-limiting example, the CNN architecture includes a stack of five convolutional blocks, with each block containing a sequence of Conv2D, ReLU (rectified linear unit) activation, batch normalization, and max pooling layers. The Conv2D layer creates multiple kernels to be convolved with the input data and produces output tensors. The kernel weights and bias vectors can be adjusted during backpropagation. As a non-limiting example, the kernel size, stride, and padding parameters may be set to 3×3, 1, “same”, respectively, for all convolutional blocks. The ReLU activation function outputs its input if it is positive and outputs zero otherwise. This adds non-linearity to the features learned by the model. The batch normalization layer normalizes each batch of data using its mean and standard deviation to facilitate backpropagation. Finally, the max pooling layer outputs the largest number within a window, such as 2×2, of its input tensor to extract increasingly generic features by down-sampling. These layers work together to allow the CNN to efficiently learn and extract useful features from the physiological measurement data, such as the SCG scalograms.

The output of the last convolutional block may be flattened and connected to two fully-connected dense units. The first unit contains a sequence of dense, rectified linear activation unit (ReLU), batch normalization, and dropout layers. The second unit includes a dense layer followed by an activation layer. However, the second unit differs based on the type of model (e.g., regression or classification). For the regression model (FIG. 4(a)), which outputs a continuous variable (e.g., Vₘₐₓ), the final dense layer may contain a single node, followed by a linear activation layer. In the classification model (FIG. 4(b)), the second unit includes a dense layer that may have several nodes. For example, the number of nodes in the final dense layer may be equal to the number of condition categories. The final activation unit outputs a probability distribution. For example, the second activation unit may be a nonlinear activation function suitable for classification tasks, such as a SoftMax activation function. Additionally or alternatively, the second activation unit may be referred to as a normalized exponential function, that produces the probabilities of the output belonging to each class. This allows the model to accurately classify the subjects based on their SCG signals or other physiological measurement data and demographic attributes.

To incorporate demographic attributes of the subjects (weight, height, age, and sex) in the deep learning model, a feedforward artificial neural network (ANN) may be created. For example, the ANN may include a multilayer perceptron (MLP), which may include two fully-connected dense layers. All of the continuous attributes (e.g., weight, height, age, etc.) can be min-max normalized. The participants' sex may be one-hot encoded (i.e. female=1, male=0) before being fed into the MLP.

In both regression (e.g., FIG. 4(a)) and classification models (e.g., FIG. 4(b)), the training parameters of MLP and CNN may be optimized using the a loss function. For example, the loss function can be calculated as the mean percentage error between the predicted and true velocities for the regression task, and the categorical cross entropy for the classification task. As a non-limiting example, the models can be implemented using Tensorflow 2.6 and Keras libraries in Python 3.8. Training and applying the model may be completed on a computer system with a GPU node. As a non-limiting example, the computer system may be equipped with a 40 GB Tesla A100 GPU.

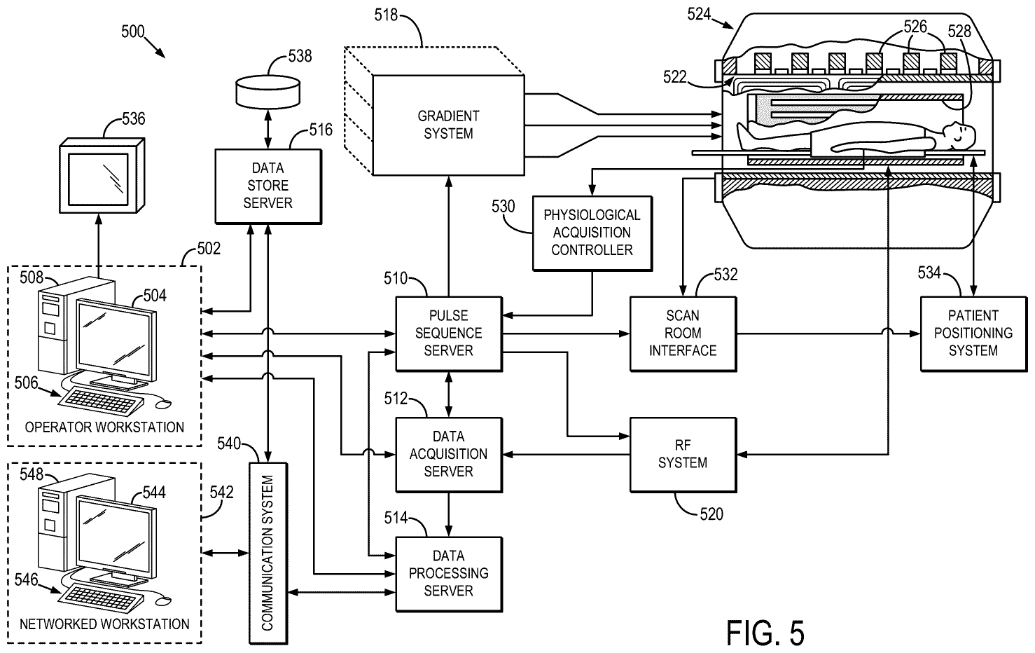

Referring particularly now to FIG. 5, an example of an MRI system 500 that can implement the methods described herein is illustrated. The MRI system 500 includes an operator workstation 502 that may include a display 504, one or more input devices 506 (e.g., a keyboard, a mouse), and a processor 508. The processor 508 may include a commercially available programmable machine running a commercially available operating system. The operator workstation 502 provides an operator interface that facilitates entering scan parameters into the MRI system 500. The operator workstation 502 may be coupled to different servers, including, for example, a pulse sequence server 510, a data acquisition server 512, a data processing server 514, and a data store server 516. The operator workstation 502 and the servers 510, 512, 514, and 516 may be connected via a communication system 540, which may include wired or wireless network connections.

The MRI system 500 also includes a magnet assembly 524 that includes a polarizing magnet 526, which may be a low-field magnet. The MRI system 500 may optionally include a whole-body RF coil 528 and a gradient system 518 that controls a gradient coil assembly 522.

The pulse sequence server 510 functions in response to instructions provided by the operator workstation 502 to operate a gradient system 518 and a radiofrequency (“RF”) system 520. Gradient waveforms for performing a prescribed scan are produced and applied to the gradient system 518, which then excited gradient coils in an assembly 522 to produce the magnetic field gradients (e.g., Gₓ, G_{y}, and G_{z}) that can be used for spatially encoding magnetic resonance signals. The gradient coil assembly 522 forms part of a magnet assembly 524 that includes a polarizing magnet 526 and a whole-body RF coil 528.

RF waveforms are applied by the RF system 520 to the RF coil 528, or a separate local coil to perform the prescribed magnetic resonance pulse sequence. Responsive magnetic resonance signals detected by the RF coil 528, or a separate local coil, are received by the RF system 520. The responsive magnetic resonance signals may be amplified, demodulated, filtered, and digitized under direction of commands produced by the pulse sequence server 510. The RF system 520 includes an RF transmitter for producing a wide variety of RF pulses used in MRI pulse sequences. The RF transmitter is responsive to the prescribed scan and direction from the pulse sequence server 510 to produce RF pulses of the desired frequency, phase, and pulse amplitude waveform. The generated RF pulses may be applied to the whole-body RF coil 528 or to one or more local coils or coil arrays.

The RF system 520 also includes one or more RF receiver channels. An RF receiver channel includes an RF preamplifier that amplifies the magnetic resonance signal received by the coil 528 to which it is connected, and a detector that detects and digitizes the received magnetic resonance signa, for example the I and Q quadrature components. As a non-limiting example, the magnitude of the received magnetic resonance signal may, therefore, be determined at a sampled point by the square root of the sum of the squares of the I and Q components:

$$M = \sqrt{( {I}^{2} + {Q}^{2} )}$$

and the phase of the received magnetic resonance signal may also be determined according to the following relationship:

$$ϕ = {tan}^{- 1} ( \frac{Q}{I} )$$

The pulse sequence server 510 may receive patient data from a physiological acquisition controller 530. By way of example, the physiological acquisition controller 530 may receive signals from a number of different sensors connected to the patient, including electrocardiograph from electrodes, or respiratory signals from a respiratory bellows or other respiratory monitoring devices. These signals may be used by the pulse sequence server 510 to synchronize, or “gate,” the performance of the scan with the subject's heartbeat or respiration.

The pulse sequence server 510 may also connect to a scan room interface circuit 532 that receives signals from various sensors associated with the condition of the patient and the magnet system. Through the scan room interface circuit 532, a patient positioning system 534 can receive commands to move the patient to desired positions during the scan.

The digitized magnetic resonance signal samples produced by the RF system 520 are received by the data acquisition server 512. The data acquisition server 512 operates in response to instructions downloaded from the operator workstation 502 to receive the real-time magnetic resonance data and provide buffer storage, so that data are not lost by data overrun. In some scans, the data acquisition server 512 passes the acquired magnetic resonance data to the data processor server 514. In scans that require information derived from acquired magnetic resonance data to control the further performance of the scan, the data acquisition server 512 may be programmed to produce such information and convey it to the pulse sequence server 510. For example, during pre-scans, magnetic resonance data may be acquired and used to calibrate the pulse sequence performed by the pulse sequence server 510. As another example, navigator signals may be acquired and used to adjust the operating parameters of the RF system 520 or the gradient system 518, or to control the view order in which k-space is sampled. In still another example, the data acquisition server 512 may also process magnetic resonance signals used to detect the arrival of a contrast agent in a magnetic resonance angiography (“MRA”) scan. For example, the data acquisition server 512 may acquire magnetic resonance data and processes it in real-time to produce information that is used to control the scan.

The data processing server 514 receives magnetic resonance data from the data acquisition server 512 and processes the magnetic resonance data in accordance with instructions provided by the operator workstation 502. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 514 are conveyed back to the operator workstation 502 for storage. Real-time images may be stored in a data base memory cache, from which they may be output to operator display 502 or a display 536. Batch mode images or selected real time images may be stored in a host database on disc storage 538. When such images have been reconstructed and transferred to storage, the data processing server 514 may notify the data store server 516 on the operator workstation 502. The operator workstation 502 may be used by an operator to archive the images, produce films, or send the images via a network to other facilities.

The MRI system 500 may also include one or more networked workstations 542. For example, a networked workstation 542 may include a display 544, one or more input devices 546 (e.g., a keyboard, a mouse), and a processor 548. The networked workstation 542 may be located within the same facility as the operator workstation 502, or in a different facility, such as a different healthcare institution or clinic.

The networked workstation 542 may gain remote access to the data processing server 514 or data store server 516 via the communication system 540. Accordingly, multiple networked workstations 542 may have access to the data processing server 514 and the data store server 516. In this manner, magnetic resonance data, reconstructed images, or other data may be exchanged between the data processing server 514 or the data store server 516 and the networked workstations 542, such that the data or images may be remotely processed by a networked workstation 542.

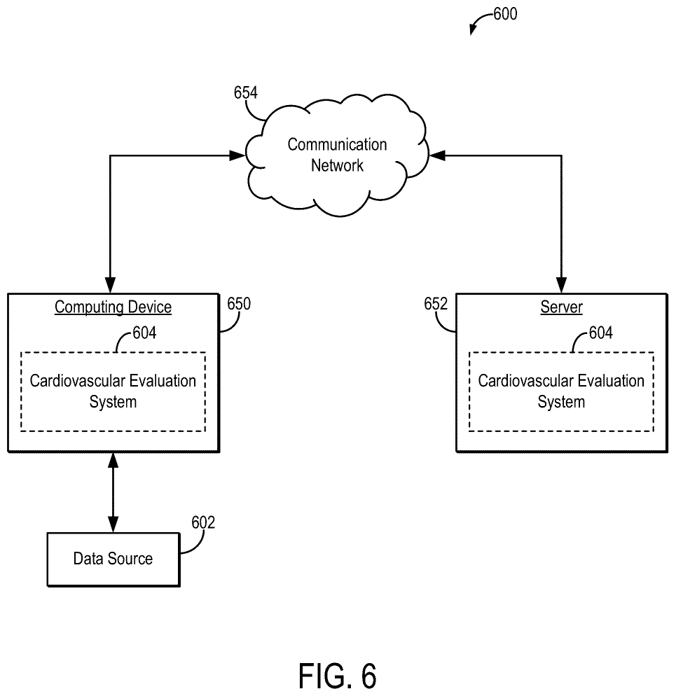

Referring now to FIG. 6, an example of a system 600 is shown, which may be used in accordance with some aspects of the systems and methods described in the present disclosure. As shown in FIG. 6, a computing device 650 can receive one or more types of data (e.g., signal evolution data, k-space data, receiver coil sensitivity data, SCG data, ECG data, PPG data, PTT data, BP data, patient data) from data source 602. In some configurations, computing device 650 can execute at least a portion of a cardiovascular evaluation system 604 to reconstruct images from magnetic resonance data (e.g., k-space data) acquired using a cardiac imaging technique. In some configurations, the cardiovascular evaluation system 604 be used to train or apply the machine learning model, as described above. The cardiovascular evaluation system may also implement an automated pipeline to provide MRI images, ground truth or estimate of Vₘₐₓ, valve classification, etc.

Additionally or alternatively, in some configurations, the computing device 650 can communicate information about data received from the data source 602 to a server 652 over a communication network 654, which can execute at least a portion of the cardiovascular evaluation system 604. In such configurations, the server 652 can return information to the computing device 650 (and/or any other suitable computing device) indicative of an output of the cardiovascular evaluation system 604.

In some configurations, computing device 650 and/or server 652 can be any suitable computing device or combination of devices, such as a desktop computer, a laptop computer, a smartphone, a tablet computer, a wearable computer, a server computer, a virtual machine being executed by a physical computing device, and so on. The computing device 650 and/or server 652 can also reconstruct images from the data.

In some configurations, data source 602 can be any suitable source of data (e.g., measurement data, images reconstructed from measurement data, processed image data, ECG data, SCG data, PPG data, PTT data, BP data), such as an MRI system, ECG device, SCG device, another sensor, another computing device (e.g., a server storing measurement data, images reconstructed from measurement data, processed image data), and so on. In some configurations, data source 602 can be local to computing device 650. For example, data source 602 can be incorporated with computing device 650 (e.g., computing device 650 can be configured as part of a device for measuring, recording, estimating, acquiring, or otherwise collecting or storing data). As another example, data source 602 can be connected to computing device 650 by a cable, a direct wireless link, and so on. Additionally or alternatively, in some configurations, data source 602 can be located locally and/or remotely from computing device 650, and can communicate data to computing device 650 (and/or server 652) via a communication network (e.g., communication network 654).

In some configurations, communication network 654 can be any suitable communication network or combination of communication networks. For example, communication network 654 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some configurations, communication network 654 can be a local area network, a wide area network, a public network (e.g., the Internet), a private or semi-private network (e.g., a corporate or university intranet), any other suitable type of network, or any suitable combination of networks. Communications links shown in FIG. 6 can each be any suitable communications link or combination of communications links, such as wired links, fiber optic links, Wi-Fi links, Bluetooth links, cellular links, and so on.

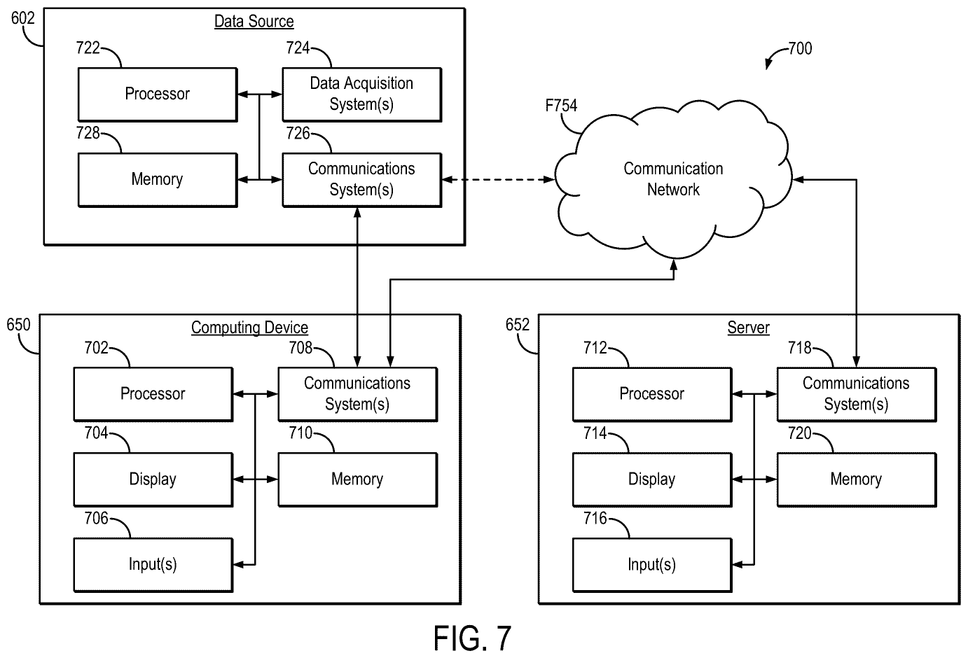

Referring now to FIG. 7, an example of hardware 700 that can be used to implement data source 602, computing device 650, and server 652 in accordance with some configurations of the systems and methods described in the present disclosure is shown.

As shown in FIG. 7, in some configurations, computing device 650 can include a processor 702, a display 704, one or more inputs 706, one or more communication systems 708, and/or memory 710. In some configurations, processor 702 can be any suitable hardware processor or combination of processors, such as a central processing unit (“CPU”), a graphics processing unit (“GPU”), and so on. In some configurations, display 704 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some configurations, inputs 706 can include any suitable input devices and/or sensors that can be used to receive user input, such as a keyboard, a mouse, a touchscreen, a microphone, and so on.

In some configurations, communications systems 708 can include any suitable hardware, firmware, and/or software for communicating information over communication network 654 and/or any other suitable communication networks. For example, communications systems 708 can include one or more transceivers, one or more communication chips and/or chip sets, and so on. In a more particular example, communications systems 708 can include hardware, firmware, and/or software that can be used to establish a Wi-Fi connection, a Bluetooth connection, a cellular connection, an Ethernet connection, and so on.

In some configurations, memory 710 can include any suitable storage device or devices that can be used to store instructions, values, data, or the like, that can be used, for example, by processor 702 to present content using display 704, to communicate with server 652 via communications system(s) 708, and so on. Memory 710 can include any suitable volatile memory, non-volatile memory, storage, or any suitable combination thereof. For example, memory 710 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some configurations, memory 710 can have encoded thereon, or otherwise stored therein, a computer program for controlling operation of computing device 650. In such configurations, processor 702 can execute at least a portion of the computer program to present content (e.g., images, user interfaces, graphics, tables), receive content from server 652, transmit information to server 652, and so on. For example, the processor 702 and the memory 710 can be configured to perform the methods described herein.

In some configurations, server 652 can include a processor 712, a display 714, one or more inputs 716, one or more communications systems 718, and/or memory 720. In some configurations, processor 712 can be any suitable hardware processor or combination of processors, such as a CPU, a GPU, and so on. In some configurations, display 714 can include any suitable display devices, such as an LCD screen, LED display, OLED display, electrophoretic display, a computer monitor, a touchscreen, a television, and so on. In some configurations, inputs 716 can include any suitable input devices and/or sensors that can be used to receive user input, such as a keyboard, a mouse, a touchscreen, a microphone, and so on.

In some configurations, communications systems 718 can include any suitable hardware, firmware, and/or software for communicating information over communication network 654 and/or any other suitable communication networks. For example, communications systems 718 can include one or more transceivers, one or more communication chips and/or chip sets, and so on. In a more particular example, communications systems 718 can include hardware, firmware, and/or software that can be used to establish a Wi-Fi connection, a Bluetooth connection, a cellular connection, an Ethernet connection, and so on.

In some configurations, memory 720 can include any suitable storage device or devices that can be used to store instructions, values, data, or the like, that can be used, for example, by processor 712 to present content using display 714, to communicate with one or more computing devices 650, and so on. Memory 720 can include any suitable volatile memory, non-volatile memory, storage, or any suitable combination thereof. For example, memory 720 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some configurations, memory 720 can have encoded thereon a server program for controlling operation of server 652. In such configurations, processor 712 can execute at least a portion of the server program to transmit information and/or content (e.g., data, images, a user interface) to one or more computing devices 650, receive information and/or content from one or more computing devices 650, receive instructions from one or more devices (e.g., a personal computer, a laptop computer, a tablet computer, a smartphone), and so on.

In some configurations, the server 652 is configured to perform the methods described in the present disclosure. For example, the processor 712 and memory 720 can be configured to perform the methods described herein.

In some configurations, data source 602 can include a processor 722, one or more data acquisition systems 724, one or more communications systems 726, and/or memory 728. In some configurations, processor 722 can be any suitable hardware processor or combination of processors, such as a CPU, a GPU, and so on. In some configurations, the one or more data acquisition systems 724 are generally configured to acquire data, images, or both, and can include an MRI system. Additionally or alternatively, in some configurations, the one or more data acquisition systems 724 can include any suitable hardware, firmware, and/or software for coupling to and/or controlling operations of an MRI system. In some configurations, one or more portions of the data acquisition system(s) 724 can be removable and/or replaceable.

Note that, although not shown, data source 602 can include any suitable inputs and/or outputs. For example, data source 602 can include input devices and/or sensors that can be used to receive user input, such as a keyboard, a mouse, a touchscreen, a microphone, a trackpad, a trackball, and so on. As another example, data source 602 can include any suitable display devices, such as an LCD screen, an LED display, an OLED display, an electrophoretic display, a computer monitor, a touchscreen, a television, etc., one or more speakers, and so on.

In some configurations, communications systems 726 can include any suitable hardware, firmware, and/or software for communicating information to computing device 650 (and, in some configurations, over communication network 654 and/or any other suitable communication networks). For example, communications systems 726 can include one or more transceivers, one or more communication chips and/or chip sets, and so on. In a more particular example, communications systems 726 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some configurations, memory 728 can include any suitable storage device or devices that can be used to store instructions, values, data, or the like, that can be used, for example, by processor 722 to control the one or more data acquisition systems 724, and/or receive data from the one or more data acquisition systems 724; to generate images from data; present content (e.g., data, images, a user interface) using a display; communicate with one or more computing devices 650; and so on. Memory 728 can include any suitable volatile memory, non-volatile memory, storage, or any suitable combination thereof. For example, memory 728 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some configurations, memory 728 can have encoded thereon, or otherwise stored therein, a program for controlling operation of medical image data source 602. In such configurations, processor 722 can execute at least a portion of the program to generate images, transmit information and/or content (e.g., data, images, a user interface) to one or more computing devices 650, receive information and/or content from one or more computing devices 650, receive instructions from one or more devices (e.g., a personal computer, a laptop computer, a tablet computer, a smartphone, etc.), and so on.

In some configurations, any suitable computer-readable media can be used for storing instructions for performing the functions and/or processes described herein. For example, in some configurations, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

### EXAMPLES

In an example study, 46 healthy subjects (20 females, age: 45.9±17.2 years) were recruited with no known history of cardiovascular disease and 31 patients with aortic valve disease (6 females, age: 32.6±20.9 years). Among the patients, 1 had a normal tricuspid aortic valve (TAV), 21 had bicuspid aortic valves (BAV), and 9 had mechanical valve implants. In addition, 12 patients were diagnosed with varying degrees of aortic stenosis (AS): 2 were mild, 6 were moderate, and 4 were severe.

Cardiac MRI assessment and two-minute SCG measurements were acquired on the same day for each subject. 4D flow MRI (spatial resolution: 1-3 mm³, temporal resolution: 30-40 ms, venc=150-375 cm/s, 1.5 T or 3 T) was performed during free breathing with navigator respiration control and full volumetric coverage of the thoracic aorta. 4D flow data analysis included preprocessing (eddy current correction, velocity anti-aliasing) and application of a previously-developed deep learning tool to automatically derive a 3D segmentation of the thoracic aorta. The 3D aorta segmentation was used to mask the 4D flow velocity data and the ascending aorta (AAo) was manually delineated from root to branching vessels. A custom code in MATLAB was used to derive a peak systolic velocity Vₘₐₓ map in the AAo (e.g., FIG. 2(a)).

A wearable SCG sensor incorporating a MEMS accelerometer was used to acquire the SCG signals. All SCG pulses were preprocessed (high-pass filtering and wavelet hard thresholding), and Signal Quality Index (SQI) based on dynamic time warping was used to identify and remove the outliers for each subject (e.g., FIGS. 3(a)-3(c)). The continuous wavelet transform (CWT) was used to obtain the time-frequency representation of the remaining SCGs. The absolute CWT coefficients resulted in the SCG scalograms (e.g., FIG. 3(d)-3(e)), which were used as the input to a convolutional neural network (CNN) to predict Vₘₐₓ in the AAo. The 4D flow MRI-derived Vₘₐₓ served as the ground truth. To incorporate the patient demographic information, a multi-layer perceptron (MLP) was added to create mixture deep neural network (DNN) models (FIGS. 4(a)-4(b)). The CNN architecture comprised 6 convolutional units, while the MLP was composed of 2 Dense layers. The outputs of MLP and CNN were concatenated and fed to a linear activation unit to generate Vₘₐₓ. To investigate the model performance, 80% of all SCG pulses (total number=6249) were used for training and the remaining 20% were reserved for testing over five random trials. To compare the results obtained using deep learning and 4D flow MRI, the linear correlation coefficient and Bland-Altman limits of agreement between two techniques were investigated.

To evaluate the performance of the models, a leave-subject-out cross-validation approach was used. This meant that for each iteration, 80% of the available SCG pulses (N=6249) were used for training the model and the remaining 20% were reserved for testing. Importantly, the SCG pulses belonging to each subject were only included in either the training or test set, but not both, in order to avoid any potential bias in the performance metrics. This process was repeated for 10 iterations, randomly selecting different subjects to be included in the training and test sets at each trial. This allowed us to verify that the reported performance metrics were not influenced by any specific patterns in the distribution of the training and test sets. For each iteration, the model was trained for 150 epochs.

To assess the agreement between the values obtained by the DNN and 4D flow MRI, correlation and Bland-Altman analyses were performed. The linear relationship between the two sets of values was determined using Pearson correlation coefficient, and the limits of agreement (LOA) were calculated using the Bland-Altman method. In Bland-Altman plots, each sample is represented by the mean of the two measurement techniques on the x-axis, and the difference between the two techniques on the y-axis. The mean difference is an estimate of the bias, the standard deviation (SD) of the difference measures the random fluctuations around the mean, and the LOA is defined as 1.96×SD. If the mean difference is significantly different from 0, based on a one-sample t-test (significance level α=0.05), this indicates the presence of a fixed bias. It is important to note that for each subject, there were multiple SCG scalograms recorded at different heartbeats. The DNN model assigned a Vₘₐₓ value to each scalogram, and the average Vₘₐₓ value over all scalograms for each subject was used as the representative value for that subject.

To investigate the performance of the classification model, receiver operating characteristic (ROC) curves were used. The ROC curves were constructed using the probabilities assigned by the model to each observation belonging to a particular valve condition group. An ROC curve plots the true positive rate (TPR) against the false positive rate (FPR) at different thresholds chosen from the predicted probabilities, and a higher ROC-AUC (area under the curve) indicates a better performance by the model. The confusion matrix obtained by testing the classification model was also used. In a confusion matrix, the diagonal elements represent the number of true positives (TP) in each category, while the off-diagonal elements at each row and column respectively show the number of false negatives (FN) and false positives (FP). In addition, the precision

$$\frac{TP}{TP + FP}$$

and recall

$$\frac{TP}{TP + FN}$$

rates in diagnosing each valve condition were calculated and compared.

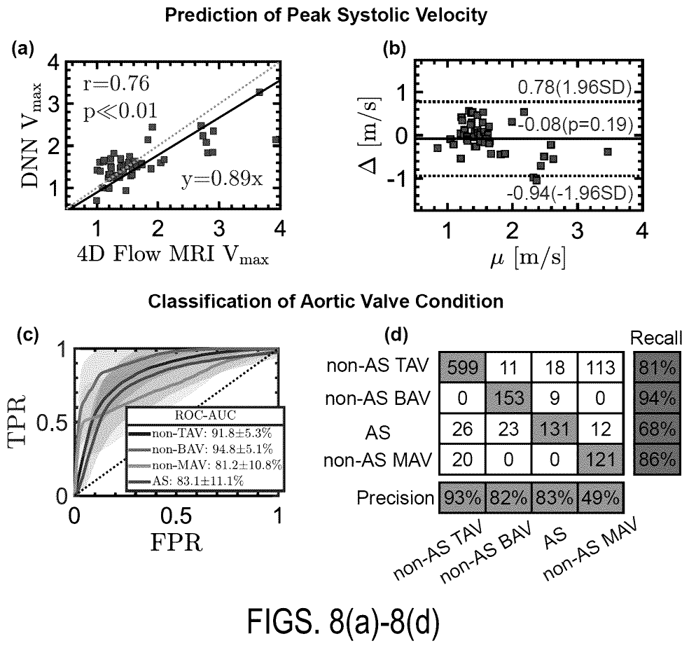

The peak systolic velocities predicted by the disclosed deep learning model were in good agreement with the velocities obtained using 4D flow MRI, as demonstrated by the low mean squared error of 0.2 m/s across ten random trials. FIG. 8(a) shows the average DNN-predicted Vₘₐₓ values versus the velocities obtained by 4D flow MRI for the test subjects. As mentioned earlier, the average Vₘₐₓ value over all scalograms for each subject was used as the representative value. The figure shows a strong linear correlation between the estimated and measured Vₘₐₓ values (y=0.89x, r=0.76, p<<0.01). Additionally, FIG. 8(b) shows the Bland-Altman plot for the Vₘₐₓ values obtained by the DNN model and 4D flow MRI measurements. This plot indicates a low, non-significant bias (−0.08 m/s, p=0.18) and moderate limits of agreement (±0.86 m/s).

FIG. 8(c) shows the results of a classification model that was trained to identify four different types of subjects based on their SCG scalograms and demographic attributes: non-AS TAV, non-AS BAV, non-AS MAV, and AS. The model was evaluated using ROC curves, which plot the true positive rate against the false positive rate. The solid lines on the ROC curves represent the mean performance of the model over 10 random iterations, while the error regions show the standard deviation of the performance across the iterations. The ROC-AUC values indicate the model's performance on each of the four classes, with values of 91.8±5.3%, 94.8±5.1%, 81.2±10.8%, and 83.1±11.1% for non-AS TAV, non-AS BAV, non-AS MAV, and AS subjects, respectively.

FIG. 8(d) shows a confusion matrix, which was used to evaluate the model's performance on the test set in one random trial. The rows of the matrix represent the ground-truth categories, while the columns represent the categories predicted by the model. The precision and recall rates indicate the model's ability to correctly classify subjects in each of the four classes. The precision rates for the four classes were 93%, 82%, 83%, and 49%, while the recall rates were 81%, 94%, 68%, and 86%.

In this study, the effectiveness of using deep learning and SCG scalograms to predict aortic peak systolic velocity and diagnose patients with various aortic valve complications was evaluated. The disclosed deep learning model accurately predicted peak systolic velocities, with MSE=0.2 m/s and r=0.76 (p<<0.01), compared to velocities obtained using 4D flow MRI.

Despite the imbalanced distribution of subjects with different types of aortic valve pathologies, high precision rates (over 82% for all categories except non-MAV) and recall rates (over 81% for all categories except AS) in the diagnosis of these conditions were achieved. The results suggest that deep learning and SCG can provide a substitute or screening tool for more advanced and costly imaging techniques such as 4D flow MRI.

Deep learning models have been previously applied to extract various types of information from SCG signals. For example, a CNN model was developed to continuously identify R-peaks from SCG signals with high sensitivity, and it was demonstrated that heart rate variability indices obtained using this model from SCG signals were in good agreement with those obtained from ECG signals. A study of 36 healthy subjects showed that deep learning could accurately map SCG signal segments to whole-body ballistocardiograms. A U-Net-based cascaded framework was also proposed for estimating respiratory rate from ECG and SCG signals. Other research has investigated the use of machine learning and deep learning for detecting heartbeats and heartbeat rates from SCG signals. In contrast to these previous works, this work examines the correlation between wearable SCG pulses and cardiac peak systolic velocity or other quantitative measures of cardiovascular function, whose ground truth may be determined using comprehensive 4D flow MRI. This study therefore expands the potential use of SCG for diagnosing cardiovascular abnormalities based on blood velocity, rather than just heart rate beats.

One potential application of the technique described in this work is to improve the accuracy of estimating aortic velocity for PC-MRI. The venc threshold will affect the ground truth measure of aortic velocity by PC-MRI. The venc threshold may be set to capture the highest expected velocity within the vessel of interest while maintaining a sufficient velocity-to-noise ratio. Moreover, in practice, though the venc value affects performance of the PC pulse sequence, it is often estimated because its optimal value is not known in advance. Thus, sometimes studies need to be repeated using different vencs to obtain the optimal results. The present disclosure provides a method to reliably estimate the optimal venc value prior to conducing an imaging study imaging, reducing MR imaging time and costs.

The patient population used as training data for the model will also affect the model performance. Thus, expanding the patient cohort to include more subjects with higher aortic velocities and different severities of AS may improve model accuracy. In practice, the training population may be tuned based on the target population for applying the model. For example, the patient population may include several patients with mild, moderate, and severe AS to expand the model in order to stratify AS severity.

### Example 2

In another example study, the 4D flow MRI provided a comprehensive assessment of cardiovascular hemodynamics such as pulsatile blood flow. This modality acquires time-resolved, three-directional measurements of three-dimensional blood flow by applying interleaved, velocity-encoding magnetic gradients along the three flow directions. However, because of utilizing advanced MRI sequences, this technique is expensive, time-consuming, and not readily available in many clinical environments. Here, it was demonstrated that seismocardiography (SCG) measurements of chest vibrations, which can be obtained using high-speed inexpensive electronics, carry sufficient information to predict the cardiovascular flow parameters such as the aortic peak velocity (Vₘₐₓ) values quantified by 4D flow MRI in fifty-eight subjects. It was also demonstrated that time-frequency distributions (scalograms) of the SCG pulses, the demographic attributes of the subjects, and deep neural networks (DNN) can together predict the Vₘₐₓ within 96.14±1.55% of the values derived from CMR measurements.

Twelve patients with aortic valve diseases (three females; age: 56.2±13.5 years) and forty-six healthy control subjects (twenty females; age: 45.9±17.2 years) with no known history of cardiovascular diseases to receive 4D flow MRI (spatial resolution: 1-3 mm³, time resolution: 30-40 ms, 1.5 T or 3 T) in addition to a two-minute-long SCG measurement on the same day were recruited. All recruitments had given informed consent within IRB supervision prior to study. Patients had either tricuspid or bicuspid aortic valve (TAV, BAV) and were presenting for routine CMR assessments. A custom-designed, wearable cardiac sensor incorporating a MEMS accelerometer capable of recording samples at a rate over 200 Hz (in particular: 1 kHz sampling rate, 2 μm/s² sensitivity) was used to acquire three-directional accelerations and to calculate their corresponding SCG pulses (an example SCG waveform is shown in FIG. 3(d)). Continuous wavelet transform (CWT) was used to obtain the time-frequency representation of each measured SCG pulse. FIG. 3(e) shows the scalogram, i.e., the absolute value of the CWT coefficients, of the signal given in FIG. 3(d). Each scalogram was transformed into an RGB image of size 64×64×3 and used them (N=6346) to a train a convolutional neural network (CNN), where each unit was composed of a sequence of a Conv2D layer, a ReLU activation layer, a Batch Normalization layer, and a MaxPooling2D layer. Furthermore, a multi-layer perceptron (MLP) composed of two densely connected layers was created to incorporate the demographic information of the patients, including weight, height, age, and sex (e.g., FIG. 4(a)). The outcome layers of the MLP and CNN were combined to predict the systolic Vₘₐₓ in the aorta. To calculate the ground-truth Vₘₐₓ along the ascending aorta (AAo), a 3D aortic segmentation was used for each subject generated automatically by a deep learning-based technique for 4D flow MRI. In addition, the AAo was manually delineated from the root to the branching vessels and performed routine volumetric analysis to obtain the Vₘₐₓ value for each patient (FIG. 2(a)). A 5-fold cross validation, i.e., 80% of the SCG scalograms and attributes were used to train the mixture DNN model while 20% of them were reserved to test the model performance over five random iterations was used. Results obtained using the DNN and 4D flow analysis are compared statistically using correlation and Bland-Altman analyses.

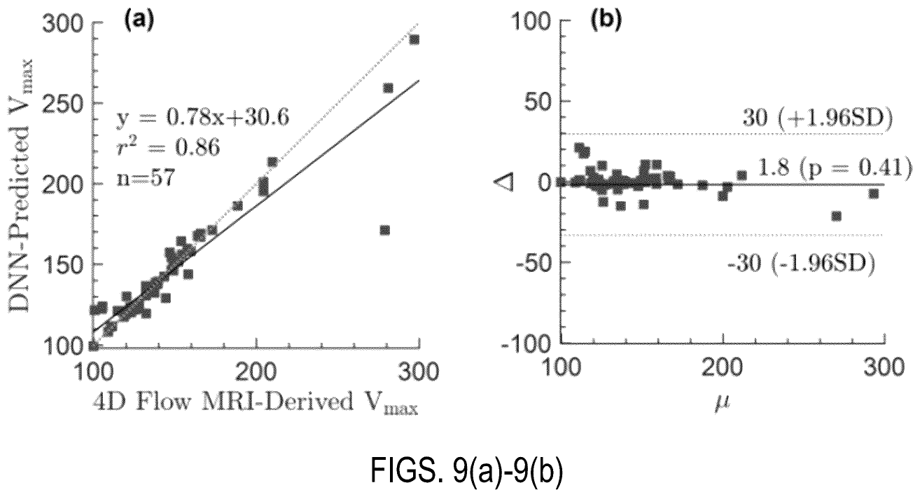

The averaged absolute percentage difference between the velocities predicted by DNN and derived using CMR was 96.14±1.55%. FIG. 9(a) demonstrates the DNN-predicted Vₘₐₓ versus the values obtained by 4D flow MRI over one iteration of the cross-validation. For each patient, the average Vₘₐₓ obtained over all the scalograms associated with that patient in the test set are given on the y-axis. It can be noticed that values obtained by these two techniques are linearly correlated (y=0.78x+30.6) with a Pearson correlation coefficient value of r²=0.86. Similar trends were observed in other instances of the cross validation. FIG. 9(b) provides the Bland-Altman plot of the values derived using DNN and CMR. It can be observed that the difference between these two techniques is not statistically significant (p=0.41).

It was demonstrated that the scalograms of the SCG pulses of chest vibrations contain sufficient information to predict the systolic Vₘₐₓ in the aorta using DNN. This study demonstrates that SCG analysis obtained using high-speed, yet significantly cheaper electronics, can be utilized for advanced cardiovascular assessment when access to CMR is not viable or as a supplement to CMR exams when advanced imaging such as 4D flow MRI is not available.

As used herein in the context of computer implementation, unless otherwise specified or limited, the terms “component,” “system,” “module,” “controller,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

As used herein, the phrase “at least one of A, B, and C” means at least one of A, at least one of B, and/or at least one of C, or any one of A, B, or C or combination of A, B, or C. A, B, and C are elements of a list, and A, B, and C may be anything contained in the Specification.

The present disclosure has described one or more preferred embodiments, and it should be appreciated that many equivalents, alternatives, variations, and modifications, aside from those expressly stated, are possible and within the scope of the invention.

1. A method for predicting cardiovascular function from physiological measurement data using machine learning, the method comprising:
(a) accessing physiological measurement data with a computer system, wherein the physiological measurement data were acquired from a subject;
(b) accessing patient data for the subject with the computer system;
(c) accessing a machine learning model that has been trained on training data to quantify cardiovascular function based on physiological measurement data and patient data;
(d) inputting the physiological measurement data and the patient data to the machine learning model using the computer system, generating cardiovascular function data as an output; and
(e) outputting the cardiovascular function data with the computer system.

2. The method of claim 1, wherein the physiological measurement data comprise seismocardiography signals.

3. The method of claim 2, wherein inputting the physiological measurement data to the neural network comprises converting the seismocardiography signals to a scalogram and inputting the scalogram to the machine learning model.

4. The method of claim 3, wherein converting the seismocardiography signals to the scalogram comprises computing a continuous wavelet transform of the seismocardiography signals.

5. The method of claim 2, further comprising accessing electrocardiography with the computer system, wherein the electrocardiography were acquired from the subject, and inputting the electrocardiography to the machine learning model as an additional input.

6. The method of claim 2, further comprising accessing a secondary physiological measurement data with the computer system, wherein the secondary physiological measurement data were acquired from the subject and comprise at least one of electrocardiography photoplethysmography, or blood pressure signals; and
processing the seismocardiography signals according to the secondary physiological measurement data.

7. The method of claim 6, wherein processing the seismocardiography signals according to the electrocardiography signals comprises gating the seismocardiography signals to obtain a seismocardiography signal for each R-R period of the electrocardiography signals.

8. The method of claim 1, wherein the physiologic measurement data comprise at least one of electrocardiography signals, photoplethysmography signals, or blood pressure recordings.

9. The method of claim 1, wherein the cardiovascular function data comprise a prediction of at least one of a peak systolic velocity (Vₘₐₓ), ejection fraction (EF), stroke volume (SV), aortic dimension, or aortic wall stiffness.

10. The method of claim 1, wherein the cardiovascular function data comprise a classification prediction of a presence of at least one of aortic valve stenosis (AS), normal tricuspid aortic valve (TAV), bicuspid aortic valve (BAV), mechanical aortic valve (MAV), mitral valve insufficiency, or cardiac output insufficiency.

11. The method of claim 1, wherein the patient data comprise demographic data, the demographic data comprising at least one of patient weight, patient height, patient age, or patient sex.

12. The method of claim 1, wherein the patient data comprise clinical data, the clinical data comprising at least one of medication usage, genetic data, history of cardiovascular conditions, or family history.

13. The method of claim 1, wherein the machine learning model comprises a neural network.

14. The method of claim 13, wherein the neural network includes one or more convolutional blocks, wherein each convolutional block includes a sequence of Conv2D, a rectified linear unit activation, batch normalization, and max pooling layers.

15. The method of claim 13, wherein the neural network includes at least a dense layer and a linear activation unit.

16. The method of claim 13, wherein the neural network includes at least a dense layer and a SoftMax activation unit.

17. The method of claim 13, wherein the neural network comprises a first neural network having an output layer having a linear activation function and a second neural network having an output layer having a nonlinear activation function, wherein the first neural network generates cardiovascular function regression data and the second neural network generates cardiovascular function classification data.

18. The method of claim 17, wherein the linear activation function is a rectified linear unit.

19. The method of claim 17, wherein the nonlinear activation function is a SoftMax function.

20. The method of claim 1, wherein accessing the physiological measurement data with the computer system includes processing the physiological measurement data with the computer system to remove at least one of interference or spurious data points from the physiological measurement data.

21. A method for training a machine learning model for evaluating cardiovascular function using ground truth data and physiological measurement data, the method comprising:
(a) accessing ground truth data with a computer system, wherein the ground truth data indicate a measure of cardiovascular function of each patient within a group of patients;
(b) accessing, with the computer system, physiological measurement data acquired from the group of patients;
(c) accessing, with the computer system, patient data from the group of patients, wherein the patient data indicate at least one of demographic or clinical data from each patient within the group of patients; and
(d) training a machine learning model using the ground truth data, the physiological measurement data, and the patient data, wherein the machine learning model comprises a multilayer perceptron and convolutional neural network to predict a cardiovascular function of a patient based on physiological measurement data and patient data acquired from the patient.

22. The method of claim 21, wherein the physiological measurement data comprises at least one of seismocardiography data, electrocardiography, or photoplethysmography data.
